(19) United States
(12) Patent Application Publication
(10) Pub. No.: US 2025/0241542 A1
(43) Pub. Date: Jul. 31, 2025

(54) POLARITY SELECTION FOR A HUB

(71) Applicant: Covidien LP, Mansfield, MA (US)

(72) Inventor: Oren Russo, Jerusalem (IL)

(21) Appl. No.: 19/012,619

(22) Filed: Jan. 7, 2025

Related U.S. Application Data
(60) Provisional application No. 63/624,992, filed on Jan. 25, 2024.

(51) Int. Cl. A61B 5/0205; A61B 5/00; A61B 5/307

(52) U.S. Cl. CPC A61B 5/02055; A61B 5/0022; A61B 5/0024; A61B 5/307

(57) ABSTRACT

Systems and methods for a patient monitoring system enable a hub (e.g., centralized computing device) to automatically receive data from varying different types of patient monitoring devices. In particular, the patient monitoring system may utilize polarity selection circuitry to automatically select (e.g., swap, switch) polarity of one or more input ports of the hub of the patient monitoring system. As such, the hub may include one or more universal ports to successfully receive or accept multiple different connectors of the varying patient monitoring devices that may include varying pin definitions.

### CROSS-REFERENCE TO RELATED APPLICATIONS

This application is related to U.S. Provisional Application No. 63/624,992, filed Jan. 25, 2024, entitled “POLARITY SELECTION FOR A HUB,” which is hereby incorporated by reference in its entirety for all purposes.

### TECHNICAL FIELD

The present technology is generally related to medical devices, particularly to computing systems, methods, and devices that improve connectivity to various patient monitoring devices and centralization of collected and displayed physiological patient parameters.

### BACKGROUND

Patient monitoring devices, such as oxygen saturation monitoring devices (e.g., near-infrared spectroscopy devices, INVOS™ devices/modules), brain monitoring devices (e.g., electroencephalogram (EEG) monitoring devices, electromyography (EMG) monitoring, and other brain monitoring such as the BIS™ monitors from Medtronic), pulse oximetry monitoring devices (e.g., Nellcor™ monitoring devices/modules), and the like, may be configured to monitor various physiological parameters of a patient. However, due to, for example, manufacturing differences, each of the various patient monitoring devices may include a separate, distinct, and/or exclusive computing device and/or display screen. To this end, multiple computing devices and/or display screens may be used to display important physiological parameters of the patient. For example, a physician may desire to view information associated with a respiratory system, a cardiac system, and a nervous system of the patient at the same time. To do so, a separate computing device, compatible with the respective sensor and/or module, along with a corresponding display screen, may be used to receive or collect and to display each of the respective desired physiological parameters of the patient. Such a system may be inefficient, especially when the patient may need to be moved to a different location (e.g., from a surgery room to a recovery room) and/or transported to a different facility. Furthermore, it may be difficult for the physician to quickly, efficiently and/or accurately assess the patient's condition as the physiological parameters may be displayed on separate and distinct display screens.

### SUMMARY

Certain embodiments commensurate in scope with the originally claimed subject matter are summarized below. These embodiments are not intended to limit the scope of the disclosure. Indeed, the present disclosure encompasses a variety of forms that may be similar to or different from the embodiments set forth below.

In an embodiment, a patient monitoring system includes a hub that includes a port that, in operation, couples to a first physiological sensor that monitors a first physiological parameter associated with a patient or to a second physiological sensor that monitors a second physiological parameter associated with the patient, wherein the second physiological parameter is different than the first physiological parameter. The patient monitoring system includes polarity selection circuitry coupled to the port, wherein the polarity selection circuitry detects one or the first physiological sensor or the second physiological sensor coupled to the port and selects an input configuration associated with the detected first or second physiological sensor. Furthermore, the patient monitoring system includes a display system coupled to the hub and configured to display the first physiological parameter or the second physiological parameter based on the selected input configuration.

In an embodiment, port of a hub including a plurality of input pins that receive a plurality of signals from a connector of a physiological sensor coupled to the port, a first signal path that transmits the plurality of signals from a first pinout arrangement to processing circuitry of the hub, and a second signal path that transmits the plurality of signals from a second pinout arrangement to the processing circuitry of the hub. The port further includes one or more switches that alternatively couple to one or more input pins of the plurality of input pins to switch between the first signal path or the second signal path, and logic controller coupled to the one or more switches that selects an input configuration associated with the one or more switches based on at least one signal of the plurality of signals.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and context of embodiments of the present disclosure without limitation to the claimed subject matter.

### BRIEF DESCRIPTION OF DRAWINGS

Advantages of the disclosed techniques may become apparent upon reading the following detailed description and upon reference to the drawings in which:

FIG. 1 is a schematic view of an embodiment of a patient monitoring system, in accordance with an aspect of the present disclosure;

FIG. 2 is a block diagram of an embodiment of the patient monitoring system of FIG. 1 including polarity selection circuitry, in accordance with an aspect of the present disclosure;

FIG. 3 is a perspective view of an embodiment of the hub of the patient monitoring system of FIG. 1, in accordance with an aspect of the present disclosure;

FIG. 4 is a front perspective view of an embodiment of the display system of the patient monitoring system of FIG. 1, in accordance with an aspect of the present disclosure;

FIG. 5 is a table diagram of an embodiment of a first pinout arrangement associated with a first connector of a first patient monitoring device and a second pinout arrangement associated with a second connector of a second patient monitoring device of the patient monitoring system of FIG. 1, in accordance with an aspect of the present disclosure;

FIG. 6 is schematic diagram of an embodiment of the polarity selection circuitry of the patient monitoring system of FIG. 1, in accordance with an aspect of the present disclosure;

FIG. 7 is a table diagram of an embodiment of a function table associated with logic control of the polarity selection circuitry of the patient monitoring system of FIG. 1, in accordance with an aspect of the present disclosure; and

FIG. 8 is a flow diagram of an embodiment of a method for selecting polarity of one or more input pins of a port of a hub via the polarity selection circuitry of the patient monitoring system of FIG. 1, in accordance with an aspect of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

Various patient monitoring systems or devices, such as oxygen saturation monitoring devices (e.g., near-infrared spectroscopy devices, INVOS™ devices), brain monitoring devices (e.g., electroencephalogram (EEG) monitoring devices, electromyography (EMG) monitoring, and other brain monitoring such as the BIS™ monitors from Medtronic), pulse oximetry monitoring devices (e.g., Nellcor™ monitoring devices), carbon dioxide (CO₂) monitoring and/or oxygen monitoring (O₂) devices, and the like, may be used to measure and display varying physiological parameters of a patient. In particular, each of these different patient monitoring systems may include a respective sensor or sensor module to measure or detect the physiological parameter as sensor data. In addition, each of the patient monitoring systems may include a respective computing device (e.g., monitor) for receiving the sensor data and, in some instances, further manipulating the sensor data or performing calculations using the sensor data to determine physiological parameters. In many cases, each of the patient monitoring systems may also include a respective display screen (e.g., display screen of the monitor) to display the physiological parameters of the patient.

To this end, multiple computing devices and/or display screens may be used by a physician and/or user to measure and display varying desired physiological parameters of the patient. For example, to determine a health status of the patient, a physician may desire to view information associated with a respiratory system of the patient, a cardiovascular system of the patient, and a nervous system of the patient at the same time. However, a separate patient monitoring system, including a sensor or sensor module, a computing device, and a corresponding display screen, may be used to receive or collect and to display physiological parameters associated with each of the respiratory system, the cardiovascular system, and the nervous system of the patient. Such an arrangement of multiple different patient monitoring systems (e.g., including the different computing devices and the respective display screens) may be inefficient and/or complex, as it may be difficult for the physician to quickly, efficiently and/or accurately assess the health status of the patient as each of the desired physiological parameters may be displayed on a separate and distinct display screen. Furthermore, each of the different patient monitoring systems may be connected (e.g., coupled) to the patient via the respective sensor and/or sensor module, and thus it may be difficult to transport with the patient along with the multiple patient monitoring systems when moving the patient to a different location (e.g., from a surgery room to a recovery room) and/or to a different facility.

Provided herein is a centralized computing device (e.g., hub) and display system that can receive data from and/or couple to multiple varying patient monitoring sensors (e.g., sensor modules) to address inefficiencies in data communication and/or presentation from physiological sensors as part of patient monitoring. Due to manufacturing differences, each of the various patient monitoring sensors may include respective connectors with a different pin definition (e.g., pin layout, pin scheme.) Systems and methods for the patient monitoring system provide improvements in the field of patient monitoring by providing a hub configured to couple to and receive data from various different kinds of patient monitoring sensors (e.g., sensor modules) that may include connectors with varying pin definitions.

To this end, the hub may be configured to receive sensor data and/or physiological parameter data from different patient monitoring sensors coupled to a patient to facilitate a collective or combined display of the physiological parameters of the patient associated with the respective data. In this way, varying physiological parameters of the patient may be displayed via a common (e.g., single, centralized, shared) display system. In particular, a physician may be able to view information related to multiple physiological systems, such as the respiratory, cardiovascular, and/or nervous system of the patient, at a single or common display location, such as a display (e.g., a centralized display, a tablet, display screen.) In addition, the patient monitoring system (e.g., the hub) may be configured to combine, manipulate, and/or perform calculations (e.g., via algorithms, machine-learning models) on the data received from the varying patient monitoring sensors. In certain cases, the patient monitoring system may operate to harmonize different data formats as part of such data combination. In addition, unification of parameters or data from the varying patient monitoring sensors into a centralized computing unit may improve synchronization between the parameters. Such unification may optimize the parameters or data through algorithms to improve quality and accuracy of the parameters, and thus improve quality and accuracy of other patient care systems or procedures, such as those used for administering and monitoring anesthesia. Furthermore, in some embodiments, varying physiological parameters may be combined via one or more algorithms to determine an overall health status or score of a patient. The overall health status or score of the patient may also be displayed via the centralized display. Thus, the patient monitoring system may enable a physician to more quickly, efficiently and/or accurately assess the health status of a patient. This may improve quality of anesthesia administering and monitoring systems or procedures, as those discussed above, by providing to an anesthesia dosage device and/or an operator of the anesthesia dosage device a more accurate assessment of a patient's health status or score during a time in which the patient may be undergoing surgery and under general anesthesia.

Furthermore, the patient monitoring system may provide for a more streamlined and portable patient monitoring system (e.g., including the hub and centralized display) that may improve mobility of a patient by eliminating the use of multiple different computing devices, each with a respective display screen, for each particular physiological parameter of the patient that is desired to be viewed and/or evaluated.

The techniques of the patient monitoring system disclosed herein enable the hub (e.g., centralized computing device) to automatically receive data from varying different types of patient monitoring sensors. In particular, the patient monitoring system may utilize polarity selection circuitry (e.g., bus switching system, USB bus system) to automatically select (e.g., swap, switch) an input configuration of one or more ports of the hub of the patient monitoring system. In particular, the polarity selection circuitry may select (e.g., swap, switch) polarity of one or more input pins of a port of the hub based on a detected connection (e.g., input, connection type) at the port. As such, the hub may include one or more ports (e.g., universal ports) to successfully couple to or accept multiple different connectors of the varying patient monitoring sensors that may include varying pin definitions. Therefore, the patient monitoring system may enable the hub to accept a wide range of varying interfaces of connectors and/or inputs that are already in use at patient monitoring and/or patient healthcare facilities, increasing overall case of use and efficiency of the patient monitoring system.

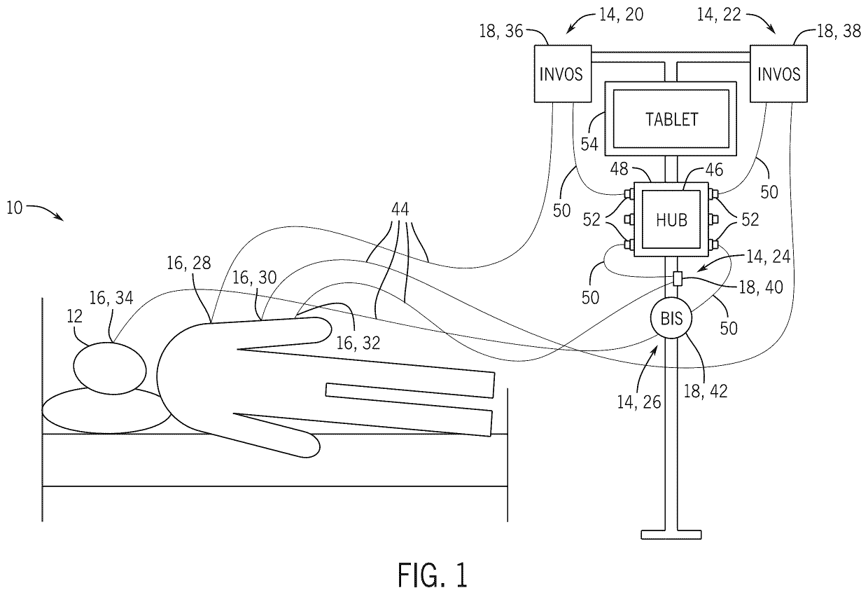

With the foregoing in mind, FIG. 1 is a schematic view of an embodiment of a patient monitoring system 10, in accordance with an aspect of the present disclosure. The patient monitoring system 10 may be configured to monitor one or more physiological parameters of a patient 12. In particular, the patient monitoring system 10 may include one or more patient monitoring sensors 14 (e.g., patient monitoring devices, patient monitors, patient physiological sensors). Each patient monitoring sensor 14 may be configured to detect, measure, and/or collect patient data associated with a particular physiological parameter. To this end, each patient monitoring sensor 14 may include one or more sensors 16 configured to couple to the patient 12 and to detect and/or measure one or more physiological parameters as sensor data. In addition, each of the patient monitoring sensors 14 may include one or more sensor modules 18 configured to receive, filter, and/or process the sensor data from the one or more respective sensors 16.

As illustrated in FIG. 1, the patient monitoring system 10 may include a first patient monitoring sensor 20, such as an oxygen saturation monitoring sensor, a second patient monitoring sensor 22, such as an additional oxygen saturation monitoring sensor, a third patient monitoring sensor 24, such as a pulse oximetry monitoring sensor, and a fourth patient monitoring sensor 26, such as an EMG monitoring sensor. It should be understood that the patient monitoring system 10 may additionally or alternatively include patient monitoring sensors that detect, measure, and/or collect other physiological parameters (e.g., as sensor data) such as CO₂ measurements (e.g., capnography), patient temperature, patient heart rate, patient respiration rate, O₂ measurements, patient blood pressure, and the like. Each of the first, second, third, and fourth patient monitoring sensors 20, 22, 24, 26 includes a respective first, second, third, and fourth sensor or set of sensors 28, 30, 32, 34, and a respective first, second, third, and fourth sensor module 36, 38, 40, 42. Each of the first, second, third, and fourth sensor(s) 28, 30, 32, 34 are coupled to the patient 12 and to the respective first, second, third, and fourth sensor modules 36, 38, 40, 42 via a respective cable 44 (e.g., line, lead).

Furthermore, the patient monitoring system 10 may include a hub 46 configured to receive data, such as sensor data and/or physiological parameters, from multiple different patent monitoring sensors 14. For example, as illustrated in FIG. 1, the hub 46 includes one or more ports 48 each configured to couple to one of the first, second, third, and fourth sensor modules 36, 38, 40, 42. The one or more ports 48 may be configured to receive data (e.g., one or more signals, sensor data, patient data, physiological parameters) from the one or more patient monitoring sensors 14. Although FIG. 1 illustrates the hub 46 with six ports, it should be understood that the hub 46 may include any suitable number of ports 48, each configured to couple to a patient monitoring sensor. As illustrated in FIG. 1, each of the first, second, third, and fourth sensor modules 36, 38, 40, 42 are coupled to a respective port 48 of the hub 46 via a respective cable 50 (e.g., line, lead) and connector 52 disposed at an end of the cable 50 and configured to couple to the one or more ports 48. As further discussed here, due to manufacturing differences, the respective connectors 52 of each of the patient monitoring sensors 14 may include a different pin definition. Thus, the patient monitoring system 10 may include or utilize the systems and techniques further discussed herein to select (e.g., adjust, swap, switch) a polarity of one or more inputs associated with the port 48 based on a type of connector 52 (e.g., a particular pin definition of the connector 52) coupled to the port 48. The patient monitoring system 10 may additionally include a display system 54 communicatively coupled to the hub 46 and configured to display patient data, such as physiological parameters, detected and/or measured by the patient monitoring sensors 14.

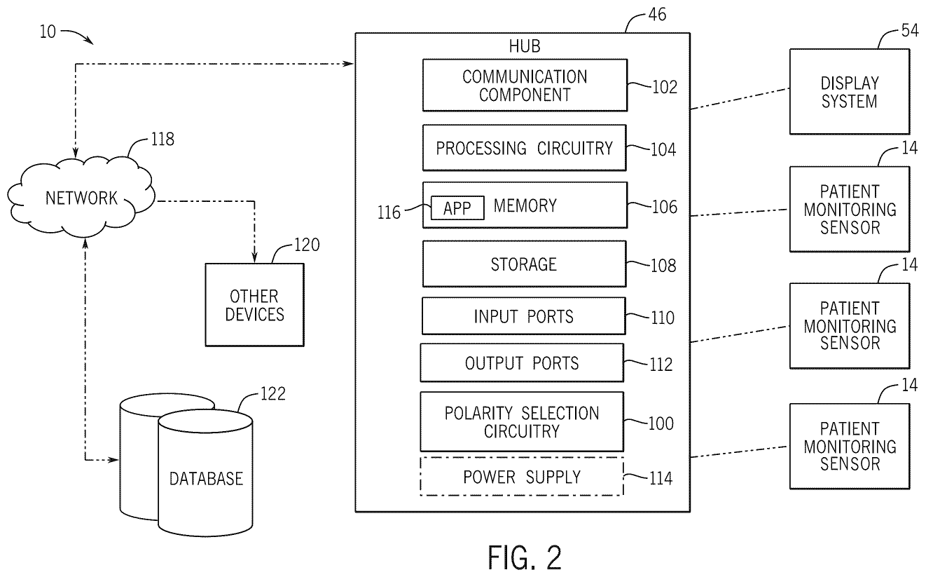

FIG. 2 is a block diagram of an embodiment of the patient monitoring system 10 of FIG. 1 including polarity selection circuitry 100, in accordance with an aspect of the present disclosure. As illustrated, the patient monitoring system 10 includes the hub 46 coupled to one or more patient monitoring sensors 14. In particular, the hub 46 may be configured to receive patient data, such as sensor data and/or physiological parameters of the patient, from the one or more patient monitoring sensors 14. Moreover, the hub 46 includes various types of components that assist the hub 46 in performing various types of tasks and operations. For example, the hub 46 includes a communication component 102, processing circuitry 104, memory 106, a storage 108, input ports 110 (e.g., ports 48), output port(s) 112, the polarity selection circuitry 100, and the like. In some embodiments, the hub 46 may include a power supply 114 configured to provide power to the hub 46. In some embodiments, the power supply 114 may be received from an external power source, such as via a plug of the hub 46 coupled to a power supply of a healthcare facility. Additionally or alternatively, the power supply 114 may include an internal rechargeable battery. In some embodiments, the power supply 114 may include a replaceable battery compartment configured to receive one or more batteries for providing power to the hub 46. In such embodiments, the power supply 114 may improve transportability of the hub 46. For example, the hub 46 may continuously provide monitoring techniques as discussed herein of a patient during transportation of the patient from a surgery room to a recovery room located at a different location within a healthcare facility.

During operation, the memory 106 may store a monitoring application 116 (e.g., patient monitoring platform), that when executed by the processing circuitry 104, monitors, stores, combines and/or manipulates patient data (e.g., received patient physiological parameters), generates and trains algorithm(s) or models based on the stored patient data, adjusts alarm threshold levels, adjusts or identifies calibration factors associated with the patient data, dynamically adjusts patient data based on the algorithms or models and the signal calibration factors, generates and/or assesses patient's health status or score (e.g., patient's physiological state) based on the patient data, determines proposed treatments or course of action based on the patient data (e.g., the patient's current condition), adjust administered remediation treatments (e.g., to the patient), identifies and detects early warnings (e.g., early warning patterns) associated with patient health status or condition based on the patient data, or any combination thereof. To this end, in some embodiments, the monitoring application 116 includes, accesses, or may be updated using a machine-learning routine that is trained based on other monitoring application data from other patient monitoring systems and/or devices within a health care facility, an institution, a local region, or the like. As such, in some embodiments, the monitoring application 116 may not directly analyze the patient data and/or learn information regarding the patient data to keep patient information confidential.

Continuing with FIG. 2, the communication component 102 may be a wireless or wired communication component that facilitates communication between the hub 46 and various other devices. In some embodiments, the communication component 102 may facilitate communication with other devices via a network 118, such as a Wireless Local Area Network (WLAN), a Local Area Network (LAN), Global area networks (e.g., the Internet), WiFi, cloud-based networks, short-range wireless signals (e.g., Bluetooth), virtual private networks (VPN), enterprise private networks (EPN), or the like. For example, the hub 46 may be communicatively coupled to the network 118, which may in turn communicatively couple to collections of other patient monitoring systems, the Internet, an Intranet system, or the like. The network 118 may facilitate communication between the hub 46 and various other data sources and/or other devices 120.

For example, the network 118 may facilitate communication between the hub 46 and a database 122 (e.g., external database) configured to store patient data, calibration information, and the like. In particular, the hub 46 may be communicatively coupled (e.g., via the network 118) to one or more databases 122 that may be configured to store patient data (e.g., historical patient data, patient health record data, patient physiological parameters), additional data associated with the monitoring application 116 (e.g., machine-learning models or algorithms, signal correction factors, alarm thresholds), or both. For example, the processing circuitry 104 may receive (e.g., retrieve) such data from the one or more databases 122. In some embodiments, the processing circuitry 104 may send a request for patient data (e.g., from a health record) associated with a patient being monitored, may receive one or more patient specific algorithms or models based on the patient data, or both.

Although the database 122 is illustrated as separate from the hub 46, in an embodiment, the database 122 may be a component within the hub 46. In an embodiment, the database 122 may be local to the hub 46 and store patient data, the additional data associated with the monitoring application 116, or both on the hub 46. In other embodiments, as described herein, the database 122 may be a cloud service or a remote database communicatively coupled to the hub 46 via the network 118.

Furthermore, the communication component 102 may facilitate wireless and/or wired communication between the hub 46 and the other devices 120. For example, the hub 46 may be configured to, via the communication component 102, send patient data to or receive patient data from a remote patient monitoring system (e.g., a surveillance monitoring system, telemetry systems), another hub and/or other patient monitoring systems, a mobile device and/or tablet, the display system 54, and the like. For example, the network 118 may facilitate communication between a patient monitoring system located on a surgery floor and an additional patient monitoring system located on an outpatient floor. In addition, in some embodiments, the communication component 102 may facilitate communication (e.g., wired or wireless) between the hub 46 and the one or more patient monitoring sensors 14.

In an embodiment, the hub 46 includes wireless functionality. For example, the hub 46 may be configured to communicatively couple to, via the communications component 102, the display system 54. In particular, the hub 46 may be transported with the patient when the patient is transferred to a subsequent location, such as different room, floor, and/or a different facility. Furthermore, the hub 46 may be configured to communicatively couple to and transmit data to existing display systems and/or display screens at the subsequent location. Thus, the wireless functionality of the hub 46 may improve portability of the device in addition to streamlining connections and improving access to and assessment of patient data.

The processing circuitry 104 may include any type of computer processor or microprocessor capable of executing computer-executable code. In addition, the processing circuitry 104 may be communicatively coupled to the one or more patient monitoring sensors 14. The processing circuitry 104 may include a processor or multiple processors that may perform the operations described below. In particular, the processing circuitry 104 receives data from the one or more patient monitoring sensors 14, such as data indicative of patient physiological parameters being measured and/or processed detected by the one or more patient monitoring sensors 14. Furthermore, the processing circuitry 104 may be configured to manipulate, perform calculations (e.g., via one or more algorithms, based on machine-learning models), analyze, combine, and/or evaluate the patient data received from the one or more patient monitoring sensors 14. For example, the processing circuitry 104 may be configured to calculate, generate, or determine an overall patient health score or status, based on the patient data. In addition, the processing circuitry 104 may be configured to utilize patient data and/or assess physiological parameters to yield patient-specific assessments, diagnostics, treatment recommendations, corrective actions, and the like.

In some embodiments, the processing circuitry 104 may be configured to receive user input such as selections for displaying patient data, selections for disabling or enabling various alarms, selections for setting certain alarm thresholds, data (e.g., patient data, health record data) input by a healthcare professional, selections for enabling or disabling administration of remedial treatments or interventions, or any combination thereof. To this end, the input ports 110 and/or output ports 112 may be interfaces that may couple to other peripheral components such as input devices (e.g., keyboard, mouse), sensors, input/output (I/O) modules, connectors, and the like.

In addition, the input ports 110 may include one or more ports 48, each port 48 configured to receive and couple to a respective connector 52 of the one or more patient monitoring sensors 14. As further discussed in more detail below, the patient monitoring system 10 may include one or more polarity selection circuitries 100 to enable the one or more ports 48 to be universal ports such that each of the one or more ports 48 may be configured to facilitate reception and/or intake of patient data (e.g., sensor data, physiological parameters) from varying types of patient monitoring sensors 14 (e.g., accept varying inputs). That is, in one example, each port 48 is configured to be coupled to a single connector 52 at one time, e.g., can couple to a first connector 52 of a first medical device type and subsequently, after decoupling from the first connector 52, is then available to couple to a second connector 52 of a second medical device type. Thus, an individual port 48 can be a multifunctional or universal port that can be coupled to one of the first connector 52 or the second connector 52, or other compatible connectors 52. In an embodiment, the first connector 52 and the second connector 52 are not simultaneously coupled to the same port 48.

To this end, the hub 46, via the one or more polarity selection circuitries 100, may be configured to select (e.g., adjust, swap, switch) an input configuration of the port based on a type of connector 52 coupled to the port 48. The input configuration of a respective port may include a pin definition or pin assignment for each of the input pins of the port. In particular, each polarity selection circuitry 100 may be configured to select (e.g., adjust, swap, switch) a polarity of one or more input pins of a respective port 48 to correlate with a polarity of one or more pins of a coupled connector 52 based on a detected connection type. More specifically, each of the one or more ports 48 of the hub 46 may be coupled to and/or may include a respective polarity selection circuitry 100. When a connector 52 is coupled to a port 48, the polarity selection circuitry 100 of the port 48 may detect (e.g., sense) a type of connection at a selection pin of the connector 52, and, based on the detected type of connection, select an input configuration of the port by selecting (e.g., adjusting, swapping, switching) a polarity of a first input pin of the port 48 to correlate with a polarity of a first pin of the connector 52, and selecting (e.g., adjusting, swapping, switching) a polarity of a second input pin of the port 48 to correlate with a polarity of a second pin of the connector 52.

The patient monitoring system 10 may enable the hub 46 to accept existing interfaces (e.g., connectors, and the like) used and installed in healthcare facilities. In addition, the ports may allow for case of use and improved efficiency of the system, as connectors of varying different types of patient monitoring systems may be inserted and/or coupled to any of the ports 48 of the hub 46, as opposed to coupled to particular or dedicated ports configured to receive specific connectors and/or patient monitoring sensors. The ports may permit arrangements with a reduced number of total ports of the system, which may in turn reduce complexity and a number of components of the hub that may be needed to support multiple ports, thereby reducing cost and increasing reliability of the patient monitoring system. In addition, the polarity selection circuitry 100 may be a hardwire component of the hub 46. Thus, selecting (e.g., adjusting, swapping, switching) the polarity of the input pins (e.g., first input pin, second input pin) of the port to correspond with the polarity of the connector pins (e.g., first output pin, second output pin) may be almost instantaneous (e.g., approximately 2 milliseconds (ms), 3 ms, 4 ms, 5 ms) in an embodiment. As such, changing or switching the polarity of the input pins may not cause a significant delay in receiving patient data from the patient monitoring sensors.

Continuing with FIG. 2, the processing circuitry 104 may be communicatively coupled to other output devices, which may include standard or special purpose computer monitors associated with the processing circuitry 104. For example, the patient monitoring system 10 includes the display system 54 configured to couple to (e.g., communicatively coupled to) the hub 46. The display system 54 may be configured to display the monitored patient physiological parameters, other patient data, current patient health status, alarms or indications, such as for recommended treatments, current treatment administration information, and the like. The display system 54 may operate as a human machine interface (HMI) (e.g., a graphical user interface (GUI)) to depict visualizations associated with software or executable code being processed by the processing circuitry 104. The display system 54 may include any suitable type of display, such as a liquid crystal display (LCD), plasma display, or an organic light emitting diode (OLED) display, for example. Additionally, in some embodiments, the display system 54 may be provided in conjunction with a touch-sensitive mechanism (e.g., a touch screen) that may function as part of a control interface for the hub 46.

Furthermore, the memory 106 and the storage 108 may be any suitable articles of manufacture that can serve as media to store processor-executable code, data, or the like. These articles of manufacture may represent computer-readable media (e.g., computer-readable instructions, any suitable form of short-term memory or long-term storage) that stores the processor-executable code used by the processing circuitry 104 to perform the presently disclosed techniques. As used herein, applications may include any suitable computer software or program that may be installed onto the hub 46 and executed by the processing circuitry 104. The memory 106 and the storage 108 may represent non-transitory (e.g., physical) computer-readable media (e.g., any suitable form of memory or storage) that may store the processor-executable code used by the processing circuitry 104 to perform various techniques described herein. For example, the memory 106 may include machine-learning algorithms or models configured to learn relationships between the patient data, calibration data, patient health condition data, or any combination thereof. In addition, the machine-learning algorithms or models may be configured to learn relationships between the physiological parameters and alarm thresholds, warnings (e.g., early warnings) or indications of patient health status, treatment administration recommendations, flow rates (e.g., of administered treatments), and the like.

As an example, the monitoring application 116 may utilize the machine-learning algorithms or models to identify early warnings associated with a patient's health status. In particular, the machine-learning algorithms or models may be configured to identify patterns in the patient data (e.g., patient physiological parameters) that indicate a critical health status may be imminent or may occur within a threshold period of time. As such, the monitoring application 116 may be able to cause the hub 46 (e.g., via the display system 54) to issue or display an early warning associated with the critical health status. Thus, a healthcare provider or the hub 46 may be able to respond preemptively to the health status of the patient and administer appropriate remedial action and/or treatments before the health status of the patient becomes critical.

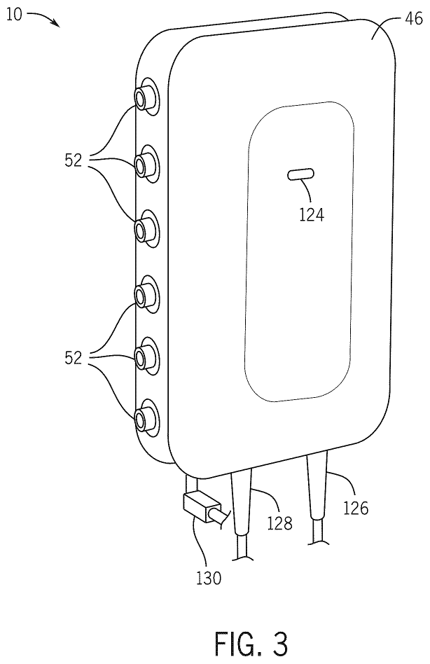

FIG. 3 is a perspective view of an embodiment of the hub 46 of the patient monitoring system 10 of FIG. 1, in accordance with an aspect of the present disclosure. In particular, the hub 46 includes the one or more ports 52 as described above with reference to FIGS. 1 and 2. As illustrated in FIG. 3, in some embodiments, the one or more ports 52 may be located on a same side of the hub device 46. In such embodiments, having the ports all located on a same or similar side may improve case of access to the one or more ports 52, and enable a more centralized (e.g., collective, consolidated, streamlined) location at which cords or cables of the one or more patient monitoring sensors may extend from the hub device 46, and thus improve efficiency and space-optimization properties of the hub device 46 (e.g., of the patient monitoring system 10). Returning to FIG. 3, the hub device 46 may additionally include an indicator light 124 configured to emit light indicating that the hub 46 is receiving power, current battery level or power level of the hub 46, that the hub 46 has a successful connection (e.g., to the network 118, to the patient monitoring sensors 14, to the display system 54, and the like), or any combination thereof. Further, the hub device 46 includes a power cable 126 configured to couple to the hub device 46 and provide power (e.g., the power supply 114) to the hub device 46 (e.g., via a power outlet), a data cable 128 configured to couple to the hub device 46 and to the display system 54. The data cable 128 may transmit data, such as patient data, sensor data, instructions or control data, image data, physiological parameters, and the like, between the hub device 46 and the display system 54. For example, the hub device 46 may transmit, via data cable 128, one or more physiological parameters that are received from the one or more patient monitoring sensors 14 and via the one or more ports 52, to the display system 54. The hub device 46 may additionally transmit instructions via the data cable 128 to the display system 54 to cause display of the one or more physiological parameters via the display system 54. The hub device 46 may additionally include a display power cable 130 configured to couple to the hub device 46 and the display system 54 (e.g., a display screen). The display power cable 130 is configured to provide power to the display system 54 from the hub device 46.

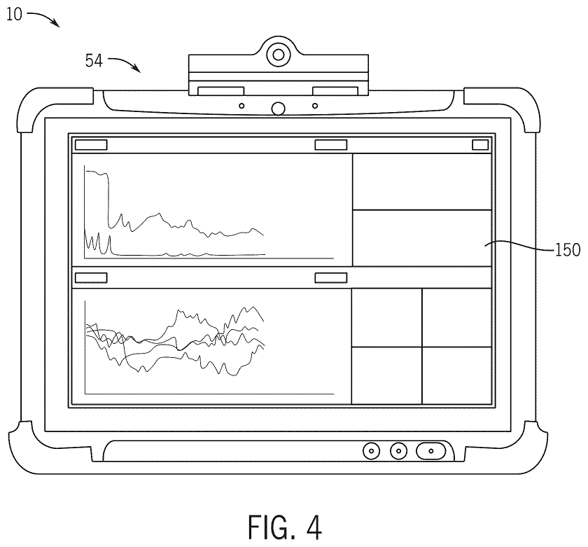

FIG. 4 is a front perspective view of an embodiment of the display system 54 of the patient monitoring system 10 of FIG. 1, in accordance with an aspect of the present disclosure. As discussed herein, the hub device 46 may transmit data, such as patient data, sensor data, physiological parameters, and the like, to the display system 54, and the display system 54 may be configured to display the data. To this end, the display system 54 may include a display screen 150 configured to present (e.g., display) the patient data, sensor data, and/or physiological parameters via one or more visual representations of the patient data, sensor data, and/or the physiological parameters. For example, the display screen 150 may present graphs, charts, physiological parameter values, or a combination thereof based on real-time data from patient monitoring sensors received via the hub device 46. Additionally or alternatively, the display screen 150 may present icons, symbols, and the like that are the visual representations of the patient data, sensor data, and/or physiological parameters received via the hub device 46.

Furthermore, the display system 54 may be configured to alternatively (e.g., adjustably) display of such visual representations based on one or more patient monitoring sensors 14 coupled to the hub device 46. For example, when the first patient monitoring sensor 20 of FIG. 1 is coupled to a first port of the hub device 46, the display system 54 may be configured to display one or more first visual representations associated with data from the first patient monitoring sensor 20 via the display screen 150. In addition, when the second patient monitoring sensor 22 is coupled to the first port of the hub device 46, the display system 54 may be configured to display one or more second visual representations associated with data from the second patient monitoring sensor 22 via the display screen 150. In some embodiments, when multiple patient monitoring sensors 14 (e.g., both the first and the second patient monitoring sensors 20, 22) are coupled to the hub device 46, the display system 54 may be configured to display (e.g., collectively display, combine display of) respective one or more visual representations for data received from each of the multiple patient monitoring sensors 14 via the display screen 150. Additionally or alternatively, the display system 54 may be configured to display, for example, based on detected input (e.g., by a user, from a patient monitoring sensor), previously stored preferences, and/or a detected type of patient monitoring device, a portion of the data from multiple monitoring sensors 14 via the display screen 150.

It should be noted that the patient monitoring system 10 should not be limited to include the components described above. Instead, the components described above with regard to the patient monitoring system 10 are examples, and the patient monitoring system 10 may include additional or fewer components relative to the illustrated embodiment.

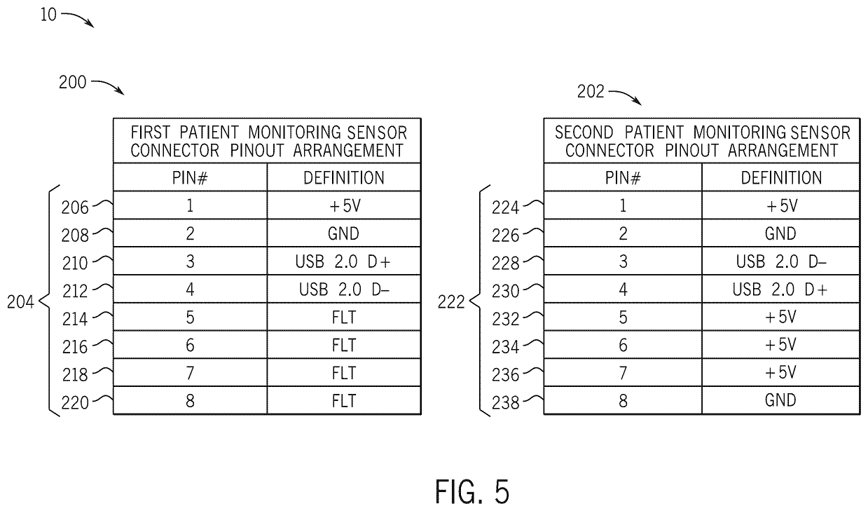

FIG. 5 is a table diagram of an embodiment of a first pinout arrangement 200 associated with a first connector (e.g., a first connector 52) of a first patient monitoring sensor and a second pinout arrangement 202 of a second connector (e.g., a second connector 52) of a second patient monitoring sensor that may be coupled to a port (e.g., a port 48) of the patient monitoring system 10 of FIG. 1, in accordance with an aspect of the present disclosure. In some embodiments, the first patient monitoring sensor may include an INVOS™ patient monitoring sensor, and the second patient monitoring sensor may include a BIS™ patient monitoring sensor. When the first connector or the second connector is coupled to the hub, each of the respective pins of the first and second connectors are configured to couple to (e.g., communicatively coupled to, mechanically couple with, contact with) a corresponding input pin of the port of the hub. For example, a first pin of the first connector and a first pin of the second connector are both configured to couple to a first input pin of the port of the hub. As such, in some embodiments, each port of the hub may include eight input pins that correspond with and/or are configured to couple to eight pins of a respective patient monitoring sensor. It should be understood that although FIG. 5 illustrates an embodiment with eight total pins, in some embodiments, a connector may include any suitable number of pins (e.g., 5, 6, 7, 9, 10, 20) to establish a connection and communicatively couple to a corresponding port of the hub. Additionally, it should be understood that the hub may include any suitable number of input pins (e.g., 5, 6, 7, 9, 10, 20) for each respective port of the hub, such that the input pins are configured to couple to a respective pin of a connector of a patient monitoring sensor.

As discussed herein, due to manufacturing differences, varying patient monitoring sensors may each include respective connectors with a separate and distinct pin definition or arrangement for the connector pins. For example, as in FIG. 5, a first connector of a first patient monitoring sensor may include the first pinout arrangement 200 that is different than the second pinout arrangement 202 associated with the second connector of a second patient monitoring sensor, though both pinout arrangements 200, 202 can be coupled to a same port as provided herein. In particular, the first patient monitoring sensor may include eight first pins 204, each pin including a particular assigned pin definition or connection. A first pin 206 may include a power definition, such as the illustrated 5 volts (V), a second pin 208 may include a ground (GND) definition, a third pin 210 may include a universal serial bus (USB) 2.0 differential data positive (D+) definition, a fourth pin 212 may include a USB 2.0 differential data negative (D−) definition, and a fifth pin 214, a sixth pin 216, a seventh pin 218, and an eighth pin 220 may each include a floating definition or connection. The floating definition or connection may include any such definition wherein the pin is not connected to a specific voltage level. For example, in some embodiments, a pin with the floating definition or connection may be not connected (e.g., unconnected pin, open pin), a weak connection (e.g., relatively low voltage), or a high impedance connection.

Continuing with FIG. 5, the second connector of the second patient monitoring sensor may additionally include eight second pins 222, each pin including a particular assigned pin definition or connection. A first pin 224 may include a power definition, such as the illustrated 5 V, a second pin 226 may include a ground definition, a third pin 228 may include the USB 2.0 D− definition, a fourth pin 230 may include the USB 2.0 D+ definition, a fifth pin 232, a sixth pin 234, and a seventh pin 236 may each include a 5 V definition or connection, and an eighth pin 238 may include an additional ground definition. As illustrated, the first pinout arrangement 200 is different than the second pinout arrangement 202.

The first pinout arrangement 200 may differ from the second pinout arrangement 202 in that the third pin 210 of the first patient monitoring sensor includes the USB 2.0 D+ definition, whereas the fourth pin 230 of the second patient monitoring sensor includes the USB 2.0 D+ definition. That is, the designation of the USB 2.0 D+ definition may occur at a third pin in one arrangement for a particular connector type while being at a fourth pin in a different arrangement for a different connector type. In addition, the fourth pin 212 of the first patient monitoring sensor includes the USB 2.0 D− definition, whereas the third pin 228 of the second patient monitoring sensor includes the USB 2.0 D− definition. Stated another way, different connectors may have switched or modified pin designations relative to one another.

Each of the first and second patient monitoring sensors is configured to communicatively couple to the hub and to send data, such as patient data, sensor data, and/or physiological parameter data to the hub via the USB 2.0 D+ and USB 2.0 D− connections. Furthermore, each of the fifth pin 214, sixth pin 216, seventh pin 218, and eighth pin 220 of the first patient monitoring sensor is different from that of the respective fifth pin 232, sixth pin 234, seventh pin 236, and eighth pin 238 of the second patient monitoring sensor. In particular, the eighth pin 220 of the first patient monitoring sensor includes the floating connection, whereas the eighth pin 238 of the second patient monitoring sensor includes the ground connection. Thus, when the respective connectors of the first and second patient monitoring sensors are coupled to the hub, the differing pin out arrangements of the first patient monitoring sensor versus the second patient monitoring sensor results in different connections, such as with different polarities (e.g., differential D+ and differential D−) and different voltage and/or impedance definitions (e.g., floating connection and ground connection), realized at respective input pins of the port of the hub.

As discussed in more detail below, to enable a successful connection, where polarities (e.g., differential D+ and differential D−) associated with the USB 2.0 assigned pins of the connector corresponds to (e.g., matches, aligns with, correlate with) polarities associated with respective pins (e.g., input pins) of the port of the hub, which enables successful transfer of data (e.g., patient data, sensor data) between the patient monitoring sensors and the hub, the hub includes the one or more polarity selection circuitries 100. Each of the one or more polarity selection circuitries 100 is configured to automatically select an input configuration of each respective port by selecting (e.g., swapping, switching) polarity of the one or more input pins of the respective port of the hub based on a detected connection type at a selection input pin (e.g., an eighth input pin) of the port. Specifically, when a particular connector is coupled to a port, the polarity selection circuitry associated with the port may detect (e.g., sense) a type of connection at a selection input pin of the port (e.g., connected to the eighth pin of the connector), and, based on the detected type of connection, select a first input configuration or a second input configuration associated with the input pins of the port. For example, the polarity selection circuitry may select the first input configuration based on detecting a first type of connection at the selection pin and may additionally select the second input configuration based on detecting a second type of connection at the selection pin. Both the first input configuration and the second input configuration cause selection of (e.g., adjustment of, swapping of, switching of) a polarity of a first input pin (e.g., connected to the third pin of the connector) of the port to correlate with (e.g., correspond to, match) a polarity of the third pin of the connector, and cause selection (e.g., adjustment of, swapping of, switching of) a polarity of a second input pin (e.g., connected to the fourth pin of the connector) of the port to correlate with a polarity of the fourth pin of the connector.

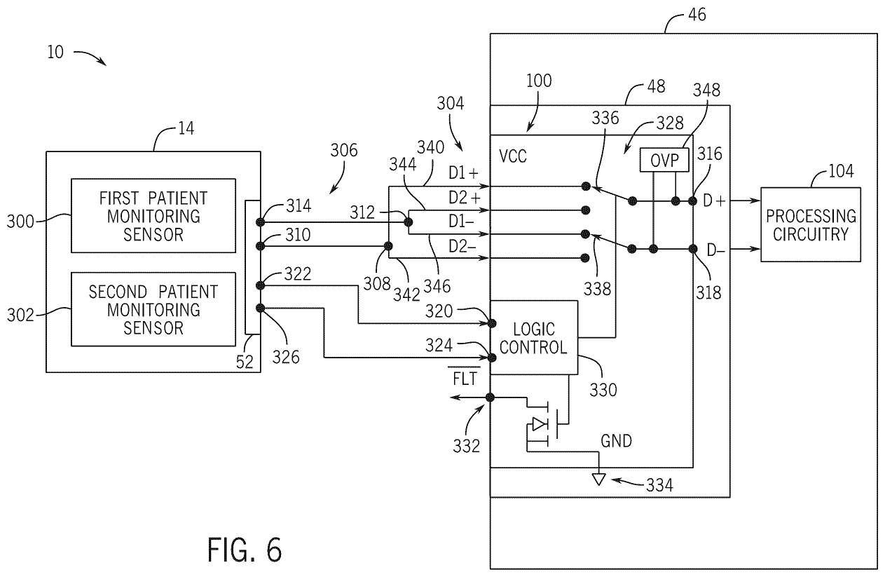

With the foregoing in mind, FIG. 6 is schematic diagram of an embodiment of the polarity selection circuitry 100 of the patient monitoring system 10 of FIG. 1, in accordance with an aspect of the present disclosure. As illustrated, the patient monitoring system 10 includes the hub 46 configured to couple to (e.g., communicatively coupled to) a patient monitoring sensor 14. In the illustrated embodiment, the hub 46 is configured to alternatively couple to either a first patient monitoring sensor 300 or a second patient monitoring sensor 302 via the port 48 (e.g., a first port) of the hub 46. The port 48 includes one or more input pins 304, each configured to couple to (e.g., receive, contact with, communicatively coupled to) a respective pin of the one or more pins 306 of the respective connector 52 of the first patient monitoring sensor 300 or the second patient monitoring sensor 302. In particular, the port 48 may include a first input pin 308 configured to couple to a first pin 310 of the connector 52, a second input pin 312 configured to couple to a second pin 314 of the connector 52. The first input pin 308 and the second input pin 312 may each be configured to alternatively couple to (e.g., connect to) a differential D+ input 316 (e.g., a differential D+ signal path) and a differential D-input 318 (e.g., a differential D-signal path) of the hub 46. Similarly, as discussed herein, the hub 46 (e.g., the processing circuitry 104) may be configured to receive data from the first patient monitoring sensor 300 or the second patient monitoring sensor 302 via the differential D+ input 316 and the differential D-input 318. In addition, the port 48 may include a third input pin 320 (e.g., a selection pin) configured to couple to a third pin 322 of the connector 52.

It should be understood that the first pin 310 of FIG. 6 may be the same as the third pins 210, 228 described in FIG. 5, the second pin 314 of FIG. 6 may be the same as the fourth pins 212, 230 described in FIG. 5, and the third pin 322 of FIG. 6 may be the same as the eighth pins 220, 238 of FIG. 5. Furthermore, in some embodiments, the port 48 may include additional input pins 304 configured to respectively couple to the above-described pins (e.g., the first pin, the second pin, the fifth pin, etc.) of the first patient monitoring sensor or the second patient monitoring sensor described with reference to FIG. 5. For example, as illustrated in FIG. 6, the port 48 may include a fourth input pin 324 configured to couple to a fourth pin 326 of the connector 52. In some embodiments, the fourth input pin 324 may be configured to receive an output-enabled input or connection via the fourth pin 326 of the connector 52.

In addition, the port 48 includes and/or is configured to couple to the polarity selection circuitry 100. The polarity selection circuitry 100 is coupled to (e.g., communicatively coupled to) the one or more input pins 304, such as, for example, the first input pin 308, the second input pin 312, and the third input pin 320 of the port 48 and is additionally coupled to the processing circuitry 104 of the hub 46. In particular, the polarity selection circuitry 100 may be coupled to the processing circuitry 104 via the differential D+ signal path 316 and the differential D-signal path 318. In some embodiments, the polarity selection circuitry 100 may be coupled in-between the port 48 and the processing circuitry 104 (e.g., configured to be an intervening component.) Furthermore, as discussed herein, the hub 46 may include a respective polarity selection circuitry 100 for each port 48 of the one or more ports 48 of the hub 46. In such embodiments, the processing circuitry 104 may be coupled to each port 48 of the one or more ports 48 of the hub 46 via a respective polarity selection circuitry 100.

In addition, the polarity selection circuitry 100 is configured to automatically select (e.g., swap, switch) polarity of the first input pin 308 and the second input pin 312 of the port 48 based on a detected connection type at the third input pin 320 (e.g., selection pin) of the port 48. In particular, the polarity selection circuitry 100 is configured to alternatively couple the first input pin 308 to either the differential D+ signal path 316 or the differential D-signal path 318, and to alternatively couple the second input pin 312 to either the differential D+ signal path 316 or the differential D-signal path 318. To this end, the polarity selection circuitry 100 may include one or more switches 328 (e.g., high speed switches), logic control 330 (e.g., digital logic control, logic controller, logic control circuitry), a floating connection 332, and a ground connection 334.

In particular, the polarity selection circuitry 100 may include a first switch 336 (e.g., high speed switch) and a second switch 338 (e.g., high speed switch.) The first switch 336 may be configured to alternatively couple (e.g., connect, enable a signal path) the differential D+ signal path 316 to either the first input pin 308 or the second input pin 312. Furthermore, the second switch 338 may be configured to alternatively couple the differential D-signal path 318 to either the first input pin 308 or the second input pin 312. The first switch 336 and/or the second switch 338 may be high speed switches. As such, the first switch 336 and/or second switch 338 may each include a transition time (e.g., transition from coupling from the first input pin 308 to the second input pin 312 and vice versa) of approximately less than 5 milliseconds (ms.) Therefore, the present embodiments provide for a port that receives various connectors of patient monitoring sensors by an approximately instantaneous selection and/or switching of the polarity of the input pins, thereby facilitating an efficient, successful connection to the respective patient monitoring sensor without a significant lag or delay in receiving data from the respective patient monitoring sensor.

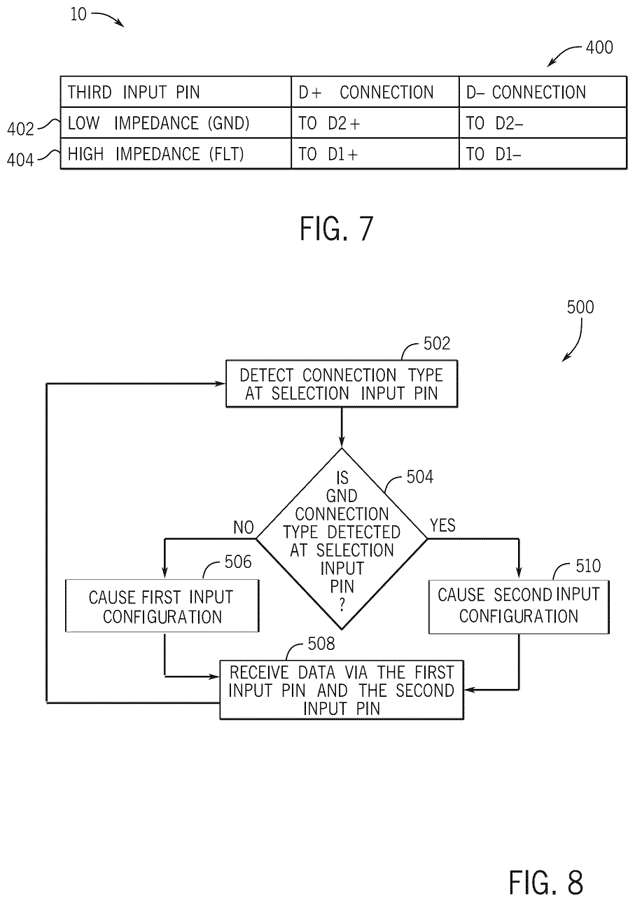

In addition, the polarity selection circuitry 100 may include the logic control 330 (e.g., controller, programmable logic controller.) The logic control 330 may include a digital logic function or table and be configured to determine a state (e.g., switch state, condition, position) of the first switch 336 and the second switch 338. FIG. 7 is a table diagram of an embodiment of a function table 400 associated with the logic control 330 of the polarity selection circuitry 100 of the patient monitoring system 10 of FIG. 1, in accordance with an aspect of the present disclosure. In particular, the logic control 330 may be configured to receive and/or detect, as an input, one or more types of connections at the third input pin 320 of the port 48 and cause, as an output, cither the first input configuration or the second input configuration. The first input configuration causing coupling of the first switch 336 to the first input pin 308 and the second switch 338 to the second input pin 312, and the second input configuration causing coupling of the first switch 336 to the second input pin 312 and the second switch 338 to the first input pin 308. For example, as illustrated in FIG. 7, when the logic control 330 receives, as input, a low impedance connection type 402 at the third pin 320 (e.g., a ground connection), the logic control 330 may cause the first switch 336 (e.g., the differential D+ signal path 316) to be coupled to the second input pin 312 (e.g., to D2+) and the second switch 338 (e.g., the differential D-signal path 318) to be coupled to the first input pin 312 (e.g., to D2−.) Alternatively, when the logic control 330 receives, as input, a high impedance connection type 404 at the third pin 320 (e.g., a floating connection), the logic control 330 may cause the first switch 336 to be connected to the first input pin 308 (e.g., to D1+) and the second switch 338 to be connected to the second input pin 312 (e.g., to D1−.)

Specifically, returning to FIG. 6, the first input pin 308 may include (e.g., may split into) a first input signal path 340 and a second input signal path 342. Similarly, the second input pin 312 may include (e.g., may split into) a third input signal path 344 and a fourth input signal path 346. As such, the first switch 336 may be configured to alternatively couple (e.g., connect, enable a signal path) the differential D+ signal path 316 to the first input signal path 340 of the first input pin 308 and the third input signal path 344 of the second input pin 312. Furthermore, the second switch 338 may be configured to alternatively couple the differential D− signal path 318 to the fourth input signal path 346 of the second input pin 312 or the second input signal path 342 of the first input pin 308. As such, the polarity selection circuitry 00 may enable the hub 46 (e.g., via the port 48) to couple to various connectors of patient monitoring sensors by automatically selecting (e.g., switching, swapping) a received polarity of the one or more input pins 304 of the port 48.

As an example, in an embodiment, the first patient monitoring sensor 300 of FIG. 6 may include an oxygen saturation monitoring sensor, such as an INVOS™ device, and include a connector with the first pinout arrangement 200 as illustrated in FIG. 3. In particular, the first patient monitoring sensor 300 may include the third pin 210 including the USB 2.0 D+ connection, the fourth pin 212 including the USB 2.0 D-connection, and the eighth pin 220 including the floating connection. In addition, the second patient monitoring sensor 302 of FIG. 6 may be a BIS™ monitoring sensor and include a connector with the second pinout arrangement 202 as illustrated in FIG. 5. In particular, the second patient monitoring sensor 302 may include the third pin 228 including the USB 2.0 D− connection, the fourth pin 230 including the USB 2.0 D+ connection, and the eighth pin 238 including the ground connection. In such an embodiment, when the connector of the first patient monitoring sensor 300 is coupled to the port 48 of the hub 46, the third pin 210 of the connector is coupled to the first input pin 308 of the port 48, the fourth pin 212 of the connector is coupled to the second input pin 312 of the port 48, and the eighth pin 220 of the connector is coupled to the third input pin 320 of the port 48. Furthermore, the polarity selection circuitry 100, via the logic control 330, may detect the floating connection at the third input pin 320 from the eighth pin 220 of the connector and cause the first switch 336 and the second switch 338 to switch to or remain in the first input configuration (e.g., a first switch state.) The first input configuration may include the first switch 336 coupling the first input signal path 340 of the first input pin 308 to the differential D+ signal path 316 and the second switch 338 coupling the fourth input signal path 346 of the second input pin 312 to the differential D− signal path 318. As such, the differential D+ signal path 316 of the port 48 may be connected to the third pin 210 of the connector, which includes the USB 2.0 D+ connection, and the differential D− signal path 318 of the port 48 may be connected to the fourth pin 212 of the connector, which includes the USB 2.0 D− connection.

In addition, when the connector of the second patient monitoring sensor 302 is coupled to the port 48 of the hub 46, the third pin 228 of the connector is coupled to the first input pin 308 of the port 48, the fourth pin 230 of the connector is coupled to the second input pin 312 of the port 48, and the eighth pin 238 of the connector is coupled to the third input pin 320. Furthermore, the polarity selection circuitry 100, via the logic control 330, may detect the ground connection at the third input pin 320 from the eighth pin 238 of the connector and cause the first switch 336 and the second switch 338 to switch to or remain in the second input configuration (e.g., a second switch state.) The second input configuration may include the first switch 336 coupling the third input signal path 344 of the second input pin 312 to the differential D+ signal path 316 and the second switch 338 coupling the second input signal path 342 of the first input pin 308 to the differential D− signal path 318. As such, the differential D+ signal path 316 of the port 48 may be connected to the fourth pin 230 of the connector, which includes the USB 2.0 D+ connection, and the differential D− signal path 318 of the port 48 may be connected to the third pin 228 of the connector, which includes the USB 2.0 D− connection. In some embodiments, the first input configuration may be a default input configuration, and when the logic control 330 detects (e.g., determines) a low impedance connection (e.g., relatively low impedance value) or ground connection type, the logic control 330 may cause the default input configuration to change (e.g., adjust, swap, switch) to the second input configuration.

In some embodiments, the polarity selection circuitry 100 may include over voltage protection 348 configured to couple to both the differential D+ signal path 316 and the differential D− signal path 318. In particular, the over voltage protection 348 may be configured to prevent excess voltage from reaching the processing circuitry 104 of the hub 46. In some embodiments, the over voltage protection 348 may be configured to open a circuit (e.g., via a series of switches) in response to a detected voltage at the differential D+ signal path 316, the differential D− signal path 318, or both exceeding a threshold voltage value.

FIG. 8 is a flow diagram of an embodiment of a method 500 for selecting polarity of one or more input pins of a port of the hub via the polarity selection circuitry of the patient monitoring system of FIG. 1, in accordance with an aspect of the present disclosure. The method 500 includes various computer-implemented steps represented by blocks that may be performed by logic control-based devices (e.g., logic control 330) described with respect to FIG. 6. It should also be noted that the method 500 may be performed by other suitable processor-based devices (e.g., processing circuitry 104) that may perform the methods described herein. Although the following description of the method 500 is described in a particular order, it should be noted that the method 500 is not limited to the depicted order; and, instead, the method 500 may be performed in any suitable order. Further, blocks/steps may be omitted and/or added to the method 500.

At block 502, a connection type is detected at a selection input pin (e.g., the third input pin 320 of FIG. 6) of a port of the hub. In particular, the polarity selection circuitry may sense (e.g., detect, determine), via the logic control, a connection type at the selection input pin. The connection type may be based on a detected impedance at the selection input pin. For example, the connection type may include a ground connection type and a floating connection type. The ground connection type may be defined as a connection with a relatively lower impedance value as compared to the floating connection type which may be defined as a connection with a relatively higher impedance value. The logic control may be configured to detect impedance at the selection input pin, and determine an input configuration (e.g., switch state) based on the detected impedance and/or the connection type. As discussed herein, the control logic may include a function table, such as the function table illustrated in FIG. 7. The logic control may determine an output of the input configuration based on an input of the detected impedance using the function table. As discussed herein, the selection input pin, such as the illustrated third input pin 320 of FIG. 6, may be configured to receive a pin, such as the described eighth pins 220, 238 of FIG. 5 and third pin 320 of FIG. 6, of a connector of a patient monitoring sensor.

With the foregoing in mind, at block 504, the logic control of the polarity selection circuitry may detect (e.g., determine) whether the connection type is a low impedance connection or ground connection type. When the detected connection type is not a low impedance connection or ground connection type (e.g., the detected connection is a high impedance connection or floating connection type), at block 506, the logic control causes, switches to, or remains in the first input configuration. As discussed herein, and with reference to FIGS. 5 and 6, the first input configuration includes the first switch 336 coupling the first input signal path 340 of the first input pin 308 to the differential D+ signal path 316 and the second switch 338 coupling the fourth input signal path 346 of the second input pin 312 to the differential D− signal path 318. As such, the differential D+ signal path 316 of the port 48 may be connected to the third pin 210 of the connector of the first patient monitoring sensor, which includes the USB 2.0 D+ connection, and the differential D− signal path 318 of the port 48 may be connected to the fourth pin 212 of the connector of the first patient monitoring sensor, which includes the USB 2.0 D− connection.

At block 508, the hub may receive data, such as sensor data, patient data, physiological parameters, and the like, from the first patient monitoring sensor via the first input pin and the second input pin.

Returning to block 405, when the detected connection type is a low impedance connection or ground connection type, at block 510, the logic control causes the second input configuration. As discussed herein, and with reference to FIGS. 5 and 6, the second input configuration includes the first switch 336 coupling the third input signal path 344 of the second input pin 312 to the differential D+ signal path 316 and the second switch 338 coupling the second input signal path 342 of the first input pin 308 to the differential D− signal path 318. As such, the differential D+ signal path 316 of the port 48 may be connected to the fourth pin 230 of the connector of the second patient monitoring sensor, which includes the USB 2.0 D+ connection, and the differential D− signal path 318 of the port 48 may be connected to the third pin 228 of the connector of the second patient monitoring sensor, which includes the USB 2.0 D− connection.

At block 508, the hub may receive data, such as sensor data, patient data, physiological parameters, and the like, from the second patient monitoring sensor via the first input pin and the second input pin. The method 500 may continue through blocks 502-510 to detect additional connection types at the selection pin (e.g., when an additional connector is coupled to the port) and cause (e.g., determine) the input configuration, such as the first input configuration or the second input configuration, based on the detected additional connection types to enable the hub to receive data from various patient monitoring sensors that may include connectors with various pinout arrangements.

The design of a USB bus system that complies with the USB 2.0 standard, particularly at high speed (e.g., 480 MHz), may present several technical challenges. As an example, high-frequency signals, such as signals in USB 2.0 high speed data transmission, are particularly sensitive to interference and signal degradation. Furthermore, the selection of conventional switching components for USB 2.0 high speed applications is limited, as very few switching components can operate at high-speed frequencies (e.g., 480 MHz) while maintaining the required signal integrity. Conventional switches often exhibit characteristics such as insertion loss, crosstalk, and parasitics, which negatively impact performance. In addition, as another example, integrating conventional switching elements into a USB bus system may introduce potential disruptions to signal continuity. As a result, in some instances, poor signal quality may require use of relatively costly cables to account for disruptions to signal continuity and poor signal quality. These disruptions can manifest as increased jitter, amplitude variations, or distortions, which can degrade the overall signal quality. Thus, realizing a USB bus system including conventional switching elements and that maintains compliance with the USB 2.0 standard's stringent requirements may be a significant challenge.

The USB Implementers Forum (USB-IF) mandates compliance with the USB eye pattern test as part of the USB 2.0 standard. This test evaluates signal integrity by analyzing jitter, signal amplitude, and overall waveform shape to ensure reliable communication.

The polarity selection circuitry of the patient monitoring system disclosed herein was tested using the USB eye pattern test, as defined by the USB-IF, to evaluate compliance with the USB 2.0 standard.

Despite the challenges discussed herein, the polarity selection circuitry successfully met the USB 2.0 standard's high-speed requirements based on the USB eye pattern test. Extensive measurements were taken during the USB eye pattern test and the measurements demonstrated compliance with the USB eye pattern test. Key results based on the measurements included minimal insertion loss and signal distortion, jitter and amplitude deviations within the acceptable range specified by the USB-IF, and successful maintenance of signal integrity, with data transmission rates at 480 MHZ.

The results of the USB eye pattern test indicated that the polarity selection circuitry of the patient monitoring system successfully overcame the performance challenges associated with implementing switching elements into a USB bus system. In addition, the polarity selection circuitry achieved robust signal integrity and compliance with the USB 2.0 standard.

The test procedure, test preparation, and testing equipment for conducting the USB eye pattern test, as defined by the USB-IF, complied with Sections 4.8 and 4.8.4 “Testing Tools,” Appendix H: USB Compliance Testing Method. The test criteria included a passing grade on the Eye-Diagram mask as defined in Keysight Infiniium SW Version 11.50.00201, Keysight Infiniium Model Number EXR254A, and Keysight Infiniium Serial Number MY63160105. Testing criteria corresponded to the standard USB eye pattern test diagram criteria, for example: USB 2.0 high-speed transmitter data rate must be 480 Mb/s+/−0.05%, EL_4: a USB 2.0 upstream facing port on a device without a captive cable must meet Template 1 transform waveform requirements measured at TP3, EL_5: a USB 2.0 upstream facing port on a device with a captive cable must meet Template 2 transform waveform requirements measured at TP2. The actual value measurement name was defined as Device Data Eye Test, and the Pass Limits were defined as Pass or Fail.

Port 1, Port 2, and Port 3, each including a respective polarity selection circuitry, were tested using the USB eye pattern test. The test results for the USB eye pattern test included Pass for Port 1, Pass for Port 2, and Pass for Port 3.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

The following examples illustrate example subject matter described herein.

Example 1. A patient monitoring system, comprising: a hub comprising a port that, in operation, couples to a first physiological sensor that monitors a first physiological parameter associated with a patient or to a second physiological sensor that monitors a second physiological parameter associated with the patient, wherein the second physiological parameter is different than the first physiological parameter; polarity selection circuitry coupled to the port, wherein the polarity selection circuitry detects one of the first physiological sensor or the second physiological sensor coupled to the port and selects an input configuration associated with the detected first or second physiological sensor; and a display system coupled to the hub and configured to display the first physiological parameter or the second physiological parameter based on the selected input configuration.

Example 2. The patient monitoring system of example 1, wherein the port comprises: a first input pin configured to couple to a first pin of a first connector associated with the first physiological sensor or of a second connector associated with the second physiological sensor; and a second input pin configured to couple to a second pin of the first connector or of the second connector.

Example 3. The patient monitoring system of example 2, wherein the polarity selection circuitry is coupled to the first input pin and the second input pin, and wherein the polarity selection circuitry comprises a switch that alternatively couples to the first input pin or the second input pin based on the selected input configuration.

Example 4. The patient monitoring system of any one of examples 2 and 3, wherein the hub comprises processing circuitry configured to receive the first physiological parameter and the second physiological parameter, and wherein the first input pin and the second input pin are alternatively coupled to the processing circuitry via a first signal path or a second signal path based on the selected input configuration, wherein the first signal path and the second signal path transmit data from the first input pin and the second input pin to the processing circuitry.

Example 5. The patient monitoring system of example 4, wherein the switch is a first switch and the polarity selection circuitry comprises a second switch that alternatively couples to the first input pin or the second input pin based on the selected input configuration.

Example 6. The patient monitoring system of example 5, wherein the selected input configuration comprises a first input configuration and a second input configuration, and wherein the first input configuration causes the first switch to couple the first signal path to the first input pin and causes the second switch to couple the second signal path to the second input pin and the second input configuration causes the first switch to couple the first signal path to the second input pin and causes the second switch to couple the second signal path to the first input pin.

Example 7. The patient monitoring system of example 6, wherein the polarity selection circuitry comprises logic control circuitry coupled to a third input pin of the port, and wherein the logic control circuitry selects the first input configuration or the second input configuration based on a detected connection type at the third input pin.

Example 8. The patient monitoring system of example 7, wherein the logic control circuitry causes the first switch to couple the first signal path to the first input pin and the second switch to couple the second signal path to the second input pin based on detecting a first connection type, and wherein the logic control circuitry causes the first switch to couple the first signal path to the second input pin and the second switch to couple the second signal path to the first input pin based on detecting a second connection type different than the first connection type.

Example 9. The patient monitoring system of example 8, wherein the first connection type comprises a first impedance value and the second connection type comprises a second impedance value, and wherein the first impedance value is higher than the second impedance value.

Example 10. The patient monitoring system of any one of examples 8 and 9, wherein the first signal path is a universal serial bus (USB) 2.0 differential data positive signal path and the second signal path is a USB 2.0 differential data negative signal path, and wherein the hub receives the first physiological parameter via the first signal path and the second signal path based on the first physiological sensor coupling to the port and receives the second physiological parameter via the first signal path and the second signal based on the second physiological sensor coupling to the port.

Example 11. A port of a hub comprising: a plurality of input pins that receive a plurality of signals from a connector of a physiological sensor coupled to the port; a first signal path that transmits the plurality of signals from a first pinout arrangement to processing circuitry of the hub; a second signal path that transmits the plurality of signals from a second pinout arrangement to the processing circuitry of the hub; one or more switches that alternatively couple to one or more input pins of the plurality of input pins to switch between the first signal path or the second signal path; and logic controller coupled to the one or more switches that selects an input configuration associated with the one or more switches based on at least one signal of the plurality of signals.

Example 12. The port of example 11, wherein the plurality of input pins comprises a first input pin, a second input pin, and a third input pin, and wherein the one or more switches comprise a first switch that alternatively couples the first signal path to the first input pin or the second input pin and a second switch that alternatively couples the second signal path to the first input pin or the second input pin.

Example 13. The port of example 12, wherein the logic controller is coupled to the third input pin and receives the at least one signal of the plurality of signals from the third input pin, and wherein the logic controller causes a first input configuration comprising the first switch coupling the first signal path to the first input pin and the second switch coupling the second signal path to the second input pin based on detecting a first impedance value associated with the at least one signal.

Example 14. The port of example 13, wherein the logic controller causes a second input configuration comprising the first switch coupling the first signal path to the second input pin and the second switch coupling the second signal path to the first input pin based on detecting a second impedance value associated with the at least one signal.

Example 15. The port of example 14, wherein the second impedance value is lower than the first impedance value.

Example 16. The port of any one of examples 11-15, wherein the first signal path is a universal serial bus (USB) 2.0 differential data positive signal path and the second signal path is a USB 2.0 differential data negative signal path, and wherein the port receives physiological data associated with a patient from the physiological sensor via the USB 2.0 differential data positive signal path and the USB 2.0 differential data negative signal path.

Example 17. A patient monitoring system, comprising: a port that, in operation, alternatively receives a first connector or a second connector different from the first connector; polarity selection circuitry coupled to the port, the polarity selection circuitry configured to select an input configuration associated with the port based on the port receiving one of the first connector or the second connector; and a display system coupled to the port and configured to receive first data via the first connector or second data via the second connector based on the selected input configuration.

Example 18. The patient monitoring system of example 17, wherein the polarity selection circuitry comprises: a first input pin configured to couple to a first pin of the first connector or of the second connector; and a second input pin configured to couple to a second pin of the first connector or of the second connector, wherein the patient monitoring system comprises processing circuitry configured to receive the first data and the second data, and wherein the first input pin and the second input pin are alternatively coupled to the processing circuitry via a first signal path or a second signal path based on the selected input configuration, wherein the first signal path and the second signal path transmit the first data or the second data from the first input pin and the second input pin to the processing circuitry.

Example 19. The patient monitoring system of example 18, wherein the polarity selection circuitry comprises: a first switch that alternatively couples the first signal path to the first input pin or the second input pin based on the selected input configuration; and a second switch that alternatively couples the second signal path to the first input pin or the second input pin based on the selected input configuration.

Example 20. The patient monitoring system of any one of examples 17-19, wherein the polarity selection circuitry comprises a third input pin, and wherein the polarity selection circuitry selects the input configuration based on an impedance at the third input pin.

Various examples of the disclosure have been described. Any combination of the described systems, operations, or functions is contemplated. These and other examples are within the scope of the following claims.

What is claimed is:

1. A patient monitoring system, comprising:
a hub comprising a port that, in operation, couples to a first physiological sensor that monitors a first physiological parameter associated with a patient or to a second physiological sensor that monitors a second physiological parameter associated with the patient, wherein the second physiological parameter is different than the first physiological parameter;
polarity selection circuitry coupled to the port, wherein the polarity selection circuitry detects one of the first physiological sensor or the second physiological sensor coupled to the port and selects an input configuration associated with the detected first or second physiological sensor; and
a display system coupled to the hub and configured to display the first physiological parameter or the second physiological parameter based on the selected input configuration.

2. The patient monitoring system of claim 1, wherein the port comprises:
a first input pin configured to couple to a first pin of a first connector associated with the first physiological sensor or of a second connector associated with the second physiological sensor; and
a second input pin configured to couple to a second pin of the first connector or of the second connector.

3. The patient monitoring system of claim 2, wherein the polarity selection circuitry is coupled to the first input pin and the second input pin, and wherein the polarity selection circuitry comprises a switch that alternatively couples to the first input pin or the second input pin based on the selected input configuration.

4. The patient monitoring system of claim 3, wherein the hub comprises processing circuitry configured to receive the first physiological parameter and the second physiological parameter, and wherein the first input pin and the second input pin are alternatively coupled to the processing circuitry via a first signal path or a second signal path based on the selected input configuration, wherein the first signal path and the second signal path transmit data from the first input pin and the second input pin to the processing circuitry.

5. The patient monitoring system of claim 4, wherein the switch is a first switch and the polarity selection circuitry comprises a second switch that alternatively couples to the first input pin or the second input pin based on the selected input configuration.

6. The patient monitoring system of claim 5, wherein the selected input configuration comprises a first input configuration and a second input configuration, and wherein the first input configuration causes the first switch to couple the first signal path to the first input pin and causes the second switch to couple the second signal path to the second input pin and the second input configuration causes the first switch to couple the first signal path to the second input pin and causes the second switch to couple the second signal path to the first input pin.

7. The patient monitoring system of claim 6, wherein the polarity selection circuitry comprises logic control circuitry coupled to a third input pin of the port, and wherein the logic control circuitry selects the first input configuration or the second input configuration based on a detected connection type at the third input pin.

8. The patient monitoring system of claim 7, wherein the logic control circuitry causes the first switch to couple the first signal path to the first input pin and the second switch to couple the second signal path to the second input pin based on detecting a first connection type, and wherein the logic control circuitry causes the first switch to couple the first signal path to the second input pin and the second switch to couple the second signal path to the first input pin based on detecting a second connection type different than the first connection type.

9. The patient monitoring system of claim 8, wherein the first connection type comprises a first impedance value and the second connection type comprises a second impedance value, and wherein the first impedance value is higher than the second impedance value.

10. The patient monitoring system of claim 8, wherein the first signal path is a universal serial bus (USB) 2.0 differential data positive signal path and the second signal path is a USB 2.0 differential data negative signal path, and wherein the hub receives the first physiological parameter via the first signal path and the second signal path based on the first physiological sensor coupling to the port and receives the second physiological parameter via the first signal path and the second signal based on the second physiological sensor coupling to the port.

11. A port of a hub comprising:
a plurality of input pins that receive a plurality of signals from a connector of a physiological sensor coupled to the port;
a first signal path that transmits the plurality of signals from a first pinout arrangement to processing circuitry of the hub;
a second signal path that transmits the plurality of signals from a second pinout arrangement to the processing circuitry of the hub;
one or more switches that alternatively couple to one or more input pins of the plurality of input pins to switch between the first signal path or the second signal path; and
logic controller coupled to the one or more switches that selects an input configuration associated with the one or more switches based on at least one signal of the plurality of signals.

12. The port of claim 11, wherein the plurality of input pins comprises a first input pin, a second input pin, and a third input pin, and wherein the one or more switches comprise a first switch that alternatively couples the first signal path to the first input pin or the second input pin and a second switch that alternatively couples the second signal path to the first input pin or the second input pin.

13. The port of claim 12, wherein the logic controller is coupled to the third input pin and receives the at least one signal of the plurality of signals from the third input pin, and wherein the logic controller causes a first input configuration comprising the first switch coupling the first signal path to the first input pin and the second switch coupling the second signal path to the second input pin based on detecting a first impedance value associated with the at least one signal.

14. The port of claim 13, wherein the logic controller causes a second input configuration comprising the first switch coupling the first signal path to the second input pin and the second switch coupling the second signal path to the first input pin based on detecting a second impedance value associated with the at least one signal.

15. The port of claim 14, wherein the second impedance value is lower than the first impedance value.

16. The port of claim 11, wherein the first signal path is a universal serial bus (USB) 2.0 differential data positive signal path and the second signal path is a USB 2.0 differential data negative signal path, and wherein the port receives physiological data associated with a patient from the physiological sensor via the USB 2.0 differential data positive signal path and the USB 2.0 differential data negative signal path.

17. A patient monitoring system, comprising:
a port that, in operation, alternatively receives a first connector or a second connector different from the first connector;
polarity selection circuitry coupled to the port, the polarity selection circuitry configured to select an input configuration associated with the port based on the port receiving one of the first connector or the second connector; and
a display system coupled to the port and configured to receive first data via the first connector or second data via the second connector based on the selected input configuration.

18. The patient monitoring system of claim 17, wherein the polarity selection circuitry comprises:
a first input pin configured to couple to a first pin of the first connector or of the second connector; and
a second input pin configured to couple to a second pin of the first connector or of the second connector, wherein the patient monitoring system comprises processing circuitry configured to receive the first data and the second data, and wherein the first input pin and the second input pin are alternatively coupled to the processing circuitry via a first signal path or a second signal path based on the selected input configuration, wherein the first signal path and the second signal path transmit the first data or the second data from the first input pin and the second input pin to the processing circuitry.

19. The patient monitoring system of claim 18, wherein the polarity selection circuitry comprises:
a first switch that alternatively couples the first signal path to the first input pin or the second input pin based on the selected input configuration; and
a second switch that alternatively couples the second signal path to the first input pin or the second input pin based on the selected input configuration.

20. The patient monitoring system of claim 19, wherein the polarity selection circuitry comprises a third input pin, and wherein the polarity selection circuitry selects the input configuration based on an impedance at the third input pin.
